SEQUENCE LISTING (61 sequences)

SEQ ID NO: 1 - DNA, 20 bp, Rattus rattus
tctccttcccattcacccac

SEQ ID NO: 2 - DNA, 28 bp, Rattus rattus
tgactccacccaaggtctccttgtttga

SEQ ID NO: 3 - DNA, 22 bp, Rattus rattus
gaatgcccagaaaagtccaagt

SEQ ID NO: 4 - DNA, 22 bp, Rattus rattus
ggcagaactctgtactgcacct

SEQ ID NO: 5 - DNA, 22 bp, Rattus rattus
tcaaattcaccttggagcctag

SEQ ID NO: 6 - DNA, 23 bp, Rattus rattus
ctgagtgtcccttaaactctccc

SEQ ID NO: 7 - DNA, 21 bp, Rattus rattus
aagtcgcttccaacctcacaa

SEQ ID NO: 8 - DNA, 22 bp, Rattus rattus
tgagatgaacatgagtgccttg

SEQ ID NO: 9 - DNA, 23 bp, Rattus rattus
agaggcagatggctgtttatctc

SEQ ID NO: 10 - DNA, 22 bp, Rattus rattus
ccctgattctggagtctgctaa

SEQ ID NO: 11 - DNA, 24 bp, Rattus rattus
cttctctctcattctggagtcagc

SEQ ID NO: 12 - DNA, 18 bp, Rattus rattus
gagtcggcttccccctct

SEQ ID NO: 13 - DNA, 23 bp, Rattus rattus
tgctctcttaacatcacctctgc

SEQ ID NO: 14 - DNA, 22 bp, Rattus rattus
agtcgcttcttgcctcagtctt

SEQ ID NO: 15 - DNA, 22 bp, Rattus rattus
tgaagatccagaccacacaacc

SEQ ID NO: 16 - DNA, 20 bp, Rattus rattus
tcatccatctccacgctgaa

SEQ ID NO: 17 - DNA, 24 bp, Rattus rattus
cagagtgactcagccacctatctc

SEQ ID NO: 18 - DNA, 24 bp, Rattus rattus
tcactcactctggcttctacctct

SEQ ID NO: 19 - DNA, 22 bp, Rattus rattus
cacatcctgatgacagaggctt

SEQ ID NO: 20 - DNA, 22 bp, Rattus rattus
ctctgaagatccaacgcacaac

SEQ ID NO: 21 - DNA, 23 bp, Rattus rattus
gctcagtgtgtcccagacttaca

SEQ ID NO: 22 - DNA, 21 bp, Rattus rattus
aacgcaagacctgaagacagc

SEQ ID NO: 23 - DNA, 24 bp, Rattus rattus
agatcaagtccagcaagctaacag

SEQ ID NO: 24 - DNA, 21 bp, Rattus rattus
cactgtacctgtgctccagca

SEQ ID NO: 25 - DNA, 24 bp, Rattus rattus
cactgatgttctgtgtgacaggtt

SEQ ID NO: 26 - DNA, 20 bp, Rattus rattus
gtgaatgggaaggagatccg

SEQ ID NO: 27 - DNA, 25 bp, Rattus rattus
ttctggcacaatcctcgcaaccact

SEQ ID NO: 28 - DNA, 27 bp, Homo sapiens
tggcctcccatctccttcatcacatct

SEQ ID NO: 29 - DNA, 21 bp, Homo sapiens
ggacaggactgaacgtcttgc

SEQ ID NO: 30 - DNA, 21 bp, Homo sapiens
ttgagcacacagagggctaca

SEQ ID NO: 31 - DNA, 22 bp, Homo sapiens
tactgcctgagcagccgcctga

SEQ ID NO: 32 - DNA, 19 bp, Homo sapiens
ggtcgctgtgtttgagcca

SEQ ID NO: 33 - DNA, 20 bp, Homo sapiens
tcctgggtccactcgtcatt

SEQ ID NO: 34 - DNA, 21 bp, Homo sapiens
tgttcccacccgaggtcgctg

SEQ ID NO: 35 - DNA, 22 bp, Homo sapiens
gatctctgcttctgatggctca

SEQ ID NO: 36 - DNA, 22 bp, Homo sapiens
ttccctgacttgcactctgaac

SEQ ID NO: 37 - DNA, 22 bp, Homo sapiens
cctgaccttgtccactctgaca

SEQ ID NO: 38 - DNA, 22 bp, Homo sapiens
ccagcaccaaccagacatctat

SEQ ID NO: 39 - DNA, 20 bp, Homo sapiens
taccgtcagcaacctggaca

SEQ ID NO: 40 - DNA, 20 bp, Homo sapiens
gggccttcagttcctctttg

SEQ ID NO: 41 - DNA, 20 bp, Homo sapiens
agctgaatgtgaacgccttg

SEQ ID NO: 42 - DNA, 22 bp, Homo sapiens
gatcacacaggtgctggagtct

SEQ ID NO: 43 - DNA, 22 bp, Homo sapiens
atccaatttctgaacacaaccg

SEQ ID NO: 44 - DNA, 23 bp, Homo sapiens
gatcacacaggagctggagtctc

SEQ ID NO: 45 - DNA, 22 bp, Homo sapiens
caagtcgcttctcacctgaatg

SEQ ID NO: 46 - DNA, 24 bp, Homo sapiens
ttctggtacagacagaccatgatg

SEQ ID NO: 47 - DNA, 22 bp, Homo sapiens
tcttcacatcaattccctggag

SEQ ID NO: 48 - DNA, 22 bp, Homo sapiens
cctctcagtacctctgtgccag

SEQ ID NO: 49 - DNA, 21 bp, Homo sapiens
gcccaagacacaaggtcacag

SEQ ID NO: 50 - DNA, 19 bp, Homo sapiens
agacctgggacatgggctg

SEQ ID NO: 51 - DNA, 19 bp, Homo sapiens
tcgacaagacccaggcatg

SEQ ID NO: 52 - DNA, 25 bp, Homo sapiens
gatggttatagtgtctccagagcaa

SEQ ID NO: 53 - DNA, 22 bp, Homo sapiens
agggagatgttcctgaagggta

SEQ ID NO: 54 - DNA, 25 bp, Homo sapiens
tctgatggatacagtgtctctcgac

SEQ ID NO: 55 - DNA, 22 bp, Homo sapiens
tctactctgaaggtgcagcctg

SEQ ID NO: 56 - DNA, 22 bp, Homo sapiens
tttcctctcactgtgacatcgg

SEQ ID NO: 57 - DNA, 22 bp, Homo sapiens
cttatttctgtgccagctcacc

SEQ ID NO: 58 - DNA, 22 bp, Homo sapiens
ctccactctcaagatccagcct

SEQ ID NO: 59 - DNA, 21 bp, Homo sapiens
ccacaaagctggaggactcag

SEQ ID NO: 60 - DNA, 22 bp, Homo sapiens
cccagttcctcatttcgtttta

SEQ ID NO: 61 - DNA, 22 bp, Homo sapiens
ctttctgctttcttgacatccg

(19) United States
(12) Patent Application Publication
(10) Pub. No.: US 2005/0019764 A1
(43) Pub. Date: Jan. 27, 2005

(54) Method for analyzing t lymphocytes with the aid of t lymphocyte receptors of an organism

(76) Inventors: Jean-Paul Soulillou, Nantes (FR); Marc-Andre Delsuc, Montpellier (FR); Marina Guillet, Nantes (FR); Sophie Brouard, Petit-Mars (FR)

Correspondence Address: FOLEY AND LARDNER;SUITE 500, 3000 K STREET NW, WASHINGTON, DC, 20007, US

(21) Appl. No.: 10/474,672

(22) PCT Filed: Mar. 28, 2002
(86) PCT No.: PCT/FR02/01087

(30) Foreign Application Priority Data
Apr. 13, 2001 (US) 60283378

(51) Int. Cl. C12Q 1/68; C12P 19/34

(52) U.S. Cl. 435/6; 435/91.2

(57) ABSTRACT

The method for analyzing the activation and motivation of T cells by analysis of T lymphocyte receptors of an organism consists in the following: determination of the number of transcripts for the gene Vβ in order to obtain first data; amplification of the region CDR3 of the T lymphocyte receptors for each of the Vβ genes associated with the organism; separation of the different lengths of the CDR3 of the T lymphocyte receptors for each of the Vβ genes associated with the organism and estimation of the proportion of transcripts of each length of the CDR3 in each Vβ family in order to obtain second data; representation of the first and second data according to the Vβ genes and the length of the regions CDR3 in a diagram with 4 variables.

The invention relates to the study of the immune system of organisms such as humans or animals, in particular to the analysis of activated or nonactivated T lymphocytes of an organism though studying its receptor.

Immune responses can be classified in two major categories: natural responses and adaptive (or specific) responses. The cells involved in the natural response use primitive and non-specific recognition systems. This type of response therefore represents the first line of defense against infections and predominates at the initial stages of infection. When the natural immunity is ineffective, adaptive immunity, which involves specific responses adapted to each microorganism, is set up. Lymphocytes form the basis of this adaptive recognition.

Two types of lymphocyte exist: B lymphocytes, responsible for humoral immunity (antibody production) and T lymphocytes, responsible for cellular immunity. The latter play a central role in biology. They recognize peptides derived from the intracellular degradation of antigens, but only when these peptides are associated with molecules carried by antigen-presenting cells, which molecules are called Major Histocompatibility Complex or MHC (also called Human Leukocyte Antigen or HLA) molecules, carried by antigen-presenting cells. For this function, T lymphocytes use a specific receptor called T lymphocyte antigen receptor or TCR.

The T cell receptor (TCR) is a heterodimer consisting of two chains: α and β. We are interested here only in the β-chain which comprises the Vβ genes (24 in humans), Dβ genes (2 in humans), Jβ genes (12 in humans) and Cβ genes (2 in humans), which are initially separated on the DNA. One of the Vβ genes becomes linked randomly to one of the Dβ, Jβ and Cβ genes so as to form a functional receptor. Thus, a large number of different TCRs can theoretically be generated (approximately 25×10⁶ different TCRs). An encounter with an antigen leads to the selection of T lymphocytes bearing a specific TCR consisting of a particular Vβ-Dβ-Jβ assembly. Recognition of the antigen occurs mainly at the level of the CDR3 (Complementary Determining Region 3) hypervariable region which is formed by the rearrangement of the Vβ, Dβ and Jβ genes and which varies in length. In a healthy individual, the transcripts encoding any Vβ family normally use all the possible lengths of the CDR3.

Up until now, analysis of the T cell receptor has mainly been carried out “qualitatively” (i.e. the search for a possible selection) by virtue of methods such as that used by the software known under the trademark Immunoscope® and which studies this receptor in terms of the length distribution for the CDR3 region, which region is thought to be the most highly involved in peptide recognition.

After amplification of this CDR3 region, of varying length, by virtue of a Cβ primer and a Vβ primer, the Immunoscope software provides the results in the form of peaks, each profile consisting of 7 to 11 peaks, i.e. of 7 to 11 different lengths for the CDR3, each separated by 3 nucleotides. Thus, when all the CDR3 lengths are represented in a given Vβ family, a Gaussian profile, characteristic of a polyclonal response, is observed. This is, for example, what is generally observed in a healthy individual. On the other hand, a CDR3 length can be represented in the majority subsequent to a particular activation which has selected particular T lymphocyte clones, and an oligo- or monoclonal “alteration” of the CDR3 region length distribution is then observed, the profiles then losing their Gaussian nature

One means for comparing the profile of a sample relative to the control profile is to use the calculation described in the article by G. Gorochov (Nat. Med., 1998, 4(2):215) which calculates the difference between the area under each peak of the control profile and the area under each peak of the profile of the sample. Percentages of alteration relative to the Gaussian profile are thus obtained, and the results are represented in the form of three-dimensional structures which make it possible to visualize the alterations associated with all the Vβ families of a given sample on the same graph. These graphs, commonly called Reperturb, appear to be flat when the repertoire is not altered and perturbed when the repertoire is altered.

Despite their advantage, these methods for qualitatively analyzing the T cell receptor do not make it possible to precisely determine the amount of Vβ mRNA affected by a selection process (and therefore indirectly the size of the pool of T cells using a given Vβ rearrangement) and therefore have a considerable limitation in that they are incapable of placing the abnormalities observed in order of size of representation.

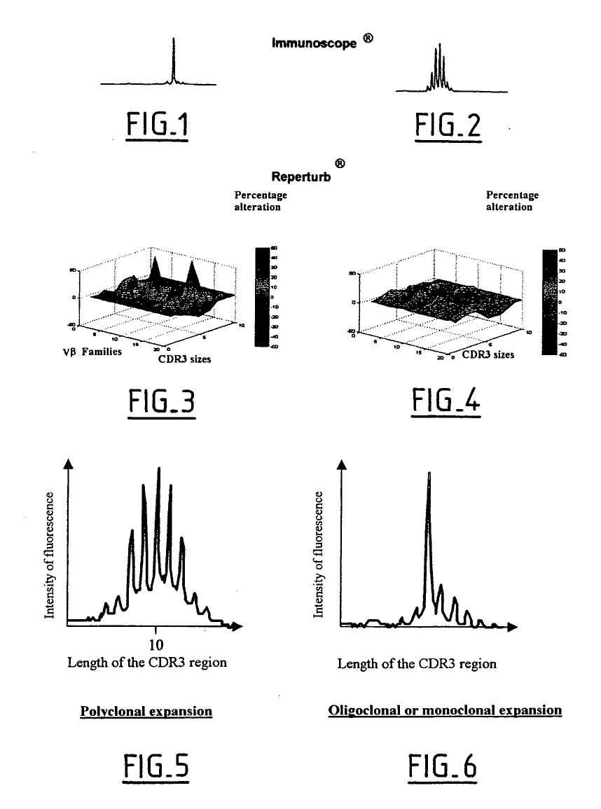

By way of example, FIG. 2 represents a Gaussian profile and FIG. 1 a monoclonal expansion obtained by Immunoscope®. Using the Reperturb method, four peaks, each corresponding to a Vβ family exhibiting a clonal expansion, appear in FIG. 3. Despite its value, this qualitative analysis alone does not make it possible to determine whether the TCRs exhibiting these expansions involve a large fraction of the Vβ transcriptome or whether they represent only a minority among the T cell pool. For example, in FIG. 3, it is not known whether the T cells bearing one of the 4 clonal expansions is represented to a greater extent than another among the total T cell population, and is therefore more particularly involved in the immune reaction studied. On the other hand, the graph remains flat when all the Vβ families exhibit a Gaussian distribution for CDR3 lengths, as shown in FIG. 4. This flat profile may be due:
to a lack of response (no T lymphocyte is involved),
to a polyclonal response due to the activation of a large number of T clones, this activation masking the presence of potential clonal expansions,
or to the activation of T cells without modification of the CDR3 length distribution. This is, for example, the case of a profile observed after stimulation with polyclonal ligands (anti-CD3 antibody, Concanavalin A, PHA, etc.) or with superantigens.

Qualitative analysis alone does not make it possible to differentiate between these three hypotheses.

The applicant was the first to use the Immunoscope® method to study allograft rejection and tolerance or, more recently, xenograft rejection and tolerance. However, although these studies were carried out on congenic rat lines (a combination which is much more “simplified” than the allogenic combination is), they revealed alterations of the “private” type (specific for an individual) in most cases, while “public” alterations (same length and same sequence for the CDR3 found in several animals) are extremely rare events. Due to this dominant “private” (and therefore apparently chaotic) profile and to the high cross-reactivity of the TCR, no reproducible information on the physiology of allorecognition can be obtained. Based on these observations and this finding, it appears that a combined analysis of the qualitative (“private”/“public”) alterations and of the quantitative estimation of the pool of T cells involved in these alterations (number of VP transcripts for each profile altered) is the only means of approaching T lymphocyte response kinetics.

Thus, our ability to evaluate and to study the dynamics and the size of the T lymphocyte response in vivo is very limited. Analysis of the TCR repertoire is an indirect reflection of the state of mobilization and of activation of T cells in a given biological situation. However, the considerable cross-reactivity of the TCR and the great diversity of the peptides processed make it difficult to use the qualitative alterations of the Vβ repertoire taken alone (for example according to the Immunoscope method) as a marker for precisely identifying and placing in order of representation the population of T cells involved in a complex immune reaction, even if the study of the repertoire is in terms of the sequences and the variations in length of the CDR3 region. Despite their value, the methods previously known for analyzing the TCR did not therefore make it possible to obtain a precise determination of the mRNA pool of T lymphocytes (relative to the overall Cβ transcriptome) using a rearrangement of given Vβ chains. In addition, accumulation of Vβ transcripts is a different parameter from the expression of the corresponding proteins at the surface of T cells since these proteins may be down-regulated after interaction with the antigen.

One of the aims of the invention is to provide a novel method of analysis which makes it possible to add a quantitative dimension, a parameter which is essential to analyzing repertoire alterations.

For the purpose of achieving this aim, we propose, according to the invention, a method for analyzing T lymphocyte receptors of an organism, which comprises the steps consisting in:
determining the number of transcripts for each Vβ gene, so as to obtain first data. This number of transcripts may then be represented, for example, in the form of crude values (number of transcripts for each Vβ family), in the form of a ratio between this number of transcripts and the number of transcripts of a reference gene, for example the HPRT (hypoxanthine phosphoribosyl transferase) gene, the latter being a gene which is present in all cells and which makes it possible to standardize the data, in the form of a percentage of each Vβ transcript among the sum of Vβ transcripts, or in the form of a measurement of the variation between the number of transcripts in a given Vβ family of a given sample and the number of transcripts of the same Vβ family of a reference sample;
amplifying the CDR3 region of the T lymphocyte receptors for each of the Vβ genes associated with the organism;
separating the various lengths of the CDR3 of the T lymphocyte receptors for each of the Vβ genes associated with the organism and estimating the proportion of transcripts for each length of the CDR3 in each Vβ family, so as to obtain second data;
representing, in a four-variable diagram, the first and second data as a function of the Vβ genes and of the length of the CDR3 regions.

Thus, the invention makes it possible to follow with precision the existence and the quantitative hierarchy of abnormalities in the length distribution for the CDR3 region during the initiation, kinetics, expansion, memory and degree of epitope spreading in a complex immune response situation (for example a pathological context), due to the fact that the two parameters (profile of alteration of the length distribution for the CDR3 region of the TCR compared to the resting situation, and quantitative evaluation of the Vβ transcripts in the pool of T cells involved) can be visualized overall, simultaneously, on the diagram.

The invention therefore provides an integrated view of the alterations in the TCR, instead of only an individual description of the alterations of each Vβ family. It is more suitable for understanding the fundamental and applied immunological events involving a T lymphocyte response (infectious diseases, auto-immune diseases, cancers, transplants, etc.). The presentation of the results is also at the same time complete, didactic and easy to understand.

The invention proposes a novel strategy for evaluating the immune response in vivo, preferably based on a computer-assisted integration of the alterations of the Vβ transcriptome of activated Tαβ0 lymphocytes.

The method of the invention combines analysis, obtained for example with the Reperturb and Immunoscope® programs, of the alteration in CDR3 length (in a given Vβ family), compared with the resting Gaussian profile, with a quantitative measurement of each Vβ transcript by quantitative PCR (for example according to the method known as TaqMan) for each Vβ family and therefore of the transcripts for all the altered TCR signals. A factorial analysis is used to define the coherence of Vβ use and of alteration thereof. The invention includes a graphic expression program which allows a general and, for example, three-dimensional view of the hierarchy of the alterations and therefore of the state of activation of the reactive T cells.

The method according to the invention may also have at least any one of the following characteristics:
at least one of the variables is represented without being associated with a dimension in space;
at least one of the variables is represented by differences in local appearance of the diagram;
the differences in local appearance are color differences;
the variable which is not associated with a dimension in space corresponds to the second data;
the first data are represented in the vertical direction;
the predetermined values correspond, for each Vβ gene, to a Gaussian distribution of the proportions of transcripts corresponding to the CDR3 regions associated with this gene;
it comprises, after determination of the first and second data, the steps consisting in choosing a Vβ gene as a function of the results from the first two steps, and in extracting from the total T cell population the T cells bearing a TCR consisting of this preselected Vβ family; and
at least some of the steps are carried out in an automated fashion.

A computer program for analyzing T lymphocyte receptors of an organism, able to control the implementation of at least the representation step of the method according to the invention is also provided for according to the invention.

Also provided for according to the invention is an installation for analyzing T lymphocyte receptors of an organism, comprising:
automated means for measuring the amounts of each Vβ gene and, optionally, the ratio of these amounts to predetermined values, so as to obtain first data;
automated means for amplifying the CDR3 region for each of the Vβ genes for the T lymphocyte receptors associated with the organism, and for measuring, for each Vβ gene, the relative amount of each CDR3 region with respect to all the CDR3 regions associated with a Vβ gene, so as to obtain second data; and
automated means for representing, in a four-variable diagram, the first and second data as a function of the Vβ genes and of the length of the CDR3 regions. This diagram allows a simplified and overall vision of the complex process of T cell mobilization during an immune reaction.

Finally, the invention provides for an information medium exhibiting a diagram analyzing the T lymphocyte receptors of an organism, comprising the following four variables:
the Vβ genes associated with the organism;
the length of the CDR3 regions of the T lymphocyte receptors;
first values representing the amount of transcripts of each Vβ gene in the form of relative or absolute amount;
second data representing, for each of the lengths of the CDR3 region in each Vβ gene, the difference between the proportion of transcripts corresponding to a CDR3 region of a Vβ gene in a given sample compared to the transcripts corresponding to the same CDR3 region of the same Vβ gene in a reference sample which is a sample exhibiting a Gaussian distribution for CDR3 lengths.

The invention may be the subject of many applications, for example in the following immunological fields:
monitoring the progression of various pathological conditions over time, such as cancers, viral diseases (AIDS, EBV infection, etc.), autoimmune diseases, etc.;
monitoring the effect of various treatments (immunotherapy, etc.);
studying the mechanisms of the immune response in various diseases and/or subsequent to various therapies;
studying the mechanisms of rejection and tolerance in transplantation;
diagnosing a “memory state”.

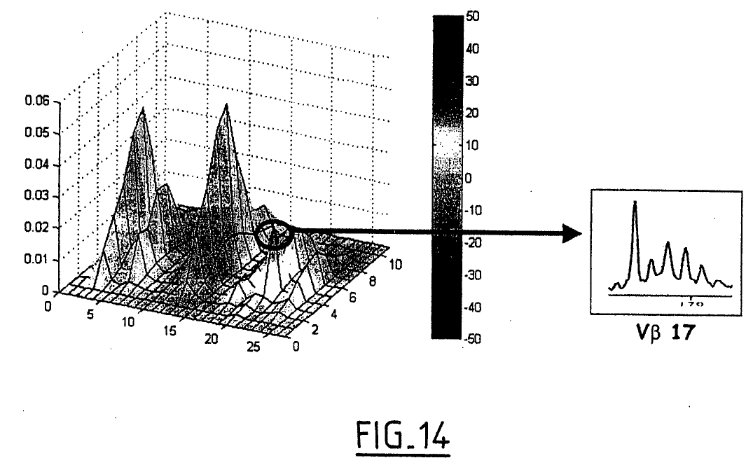

Some examples of applications are proposed more precisely below:
monitoring the progression of the disease and searching for a possible correlation between the regression or the worsening of the disease and the size of the “spreading”, which corresponds to the fact that the T clone selection will evolve over time due to the recognition of novel antigenic structures by molecular mimicking;
monitoring the evolution, over time, of the T cell repertoire in patients who may or may not be exhibiting a continuous regression of the disease after immunotherapy. The invention will make it possible to monitor both the effect of the therapy and the development of the immune response against the disease;
detecting specific markers associated with the progression of the disease. For example, an increase in clonal alterations, an increase in the number of Vβ families exhibiting particular alterations and/or a significant increase in Vβ messengers in the blood, taken over considerable periods of time, may make it possible to study the “epitope spreading”. Such differences will make it possible to distinguish the T responses before and after a treatment from the responses more highly involved in the regression of the disease due to the therapy;
distinguishing between early immune processes and those which have been set up for a long time in the case of autoimmune diseases for example, and between the immune processes involved in various forms of the same disease;
comparing the graphic representations provided via the invention with the results obtained with conventional techniques of diagnosis and of immunoanalysis carried out by other methods (for example: monitoring of T cells by tetramers, monitoring of the production of cytokines by the method known as Elispot) and possibility of correlating the evolution of the appearance and disappearance of T clones over time with the clinical progression of the patient, so as to obtain important information on the symptomatology and the immune processes involved in the disease.
Various modifications may be observed after treatment of a disease (disappearance of clones, modification of quantitative values, modification of overall profile topology, etc.). These modifications may indicate a down-regulation, the deletion of autoreactive clones or else an evolution of the T cells involved. This symptomatology may vary according to the strategy used. Specifically, some agents can induce apoptosis of autoreactive cells, while cytotoxic drugs such as cyclosporin or mitoxantrone can cause aspecific modifications. Theoretically, the profiles for the patients obtained using the invention might therefore influence the decision regarding treatment. The invention may also be useful for establishing criteria for tolerance in patients apparently lacking clinical symptoms;
possibility of identifying, peripherally, specific representations of certain diseases. Similarly, the possibility of superimposing the graphs obtained from the blood and those observed in the transplant is envisioned (for example for detecting signs of chronic rejection via a blood sample);
The analysis according to the invention, on samples from patients, may make it possible to define a new parameter for selecting epitopes which are candidates for a more effective immunotherapy (antitumor immunotherapy);
isolation of T cells revealed by the combination of the first and second data as being particularly involved in the immune reaction studied. An analysis of the specificity and of the functionality of these isolated T cells may then be carried out. This identification then isolation of T cells carrying a particular TCR makes it possible to carry out functional or phenotypic studies for example, not on the total T cell population but only on the T cells involved in the immune reaction studied. This improved “gene searching” approach will make it possible to identify novel strategies for immunotherapy. By way of example, see FIG. 14.

The stakes involved for many immunizing procedures are to develop a T response and therefore to measure it. In this respect, the invention represents a novel, potentially very useful, addition. Furthermore, studying the topology of the repertoires represented may be important for detecting the specific “signatures” of a pathological process in an individual. Specifically, the invention can be used, for example, to analyze the progression of the pathological process in patients and/or to influence the decision regarding a treatment.

In the field of pharmaceutical and medical research, the invention will be of use for understanding and monitoring pathological conditions and/or treatments in various fields such as:
virology;
cancerology;
autoimmune diseases;
treatment of allergies (monitoring of desensitizations);
transplantation of organs, of tissues or of marrow, anticipation of the occurrence of acute or chronic rejection, search for regulating clones in transplant patients in order to determine whether these clones are liable to be tolerant with respect to their transplant in the absence of any immunosuppressor treatment, etc.

In medical research, the invention will be of use, for example, as a clinical tool to aid with diagnosis (viral diseases, autoimmune diseases, cancer) and for monitoring therapies (vaccines, cell therapies, gene therapies).

The invention can be used on animals in the context of research projects (animal tests, preclinical analyses, etc.) and on humans in the context of treatment assessment.

Other characteristics and advantages of the invention will emerge further in the following description of several preferred embodiments given by way of nonlimiting examples, with reference to the attached drawings:

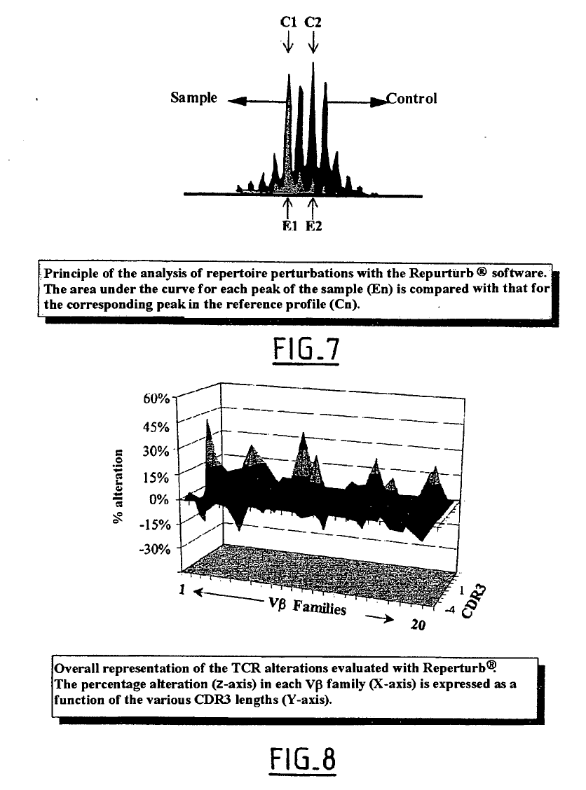

FIGS. 1 to 8 are diagrams relating to the study of T lymphocytes, and obtained with techniques of the prior art;

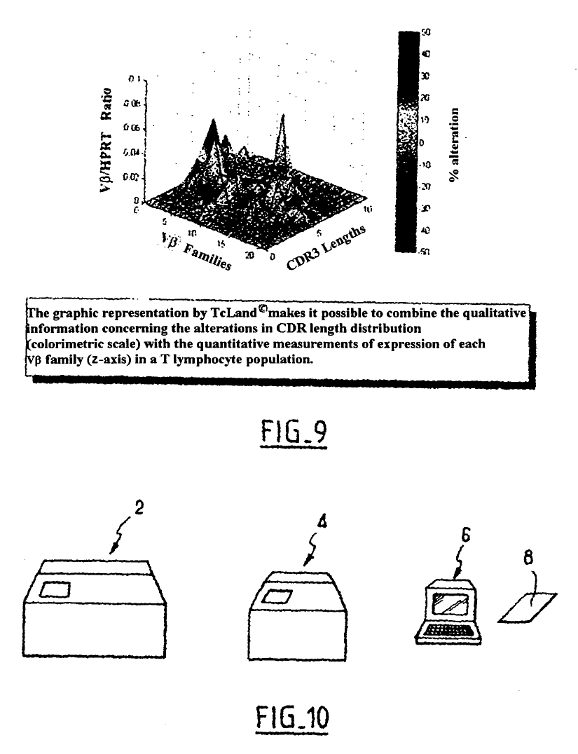

FIGS. 9 and 11 to 13 are diagrams obtained with the method according to the invention;

FIG. 10 is a diagrammatic representation of the means for implementing the invention and of the medium obtained; and

FIG. 14 represents an example of sorting. In this example, a TcLandscape profile was obtained from a patient suffering from multiple sclerosis. On the graph, it is observed that the Vβ17 family is altered and that the number of Vβ17 transcripts is large. The T cells bearing a TCR composed of Vβ17 genes were isolated by flow cytometry and functional studies were carried out on these isolated cells. Particularly high transcription of the cytokines of I1-6, I1-8 and TNFα is observed in these cells. This accumulation is not observed in the T cells bearing a Vβ17+ TCR from a healthy individual. Furthermore, these Vβ17+ cells isolated from the patient exhibit an activated phenotype.

### EXAMPLES

A first embodiment of the method according to the invention will be described in the context of the cellular response in xenotransplantation.

In transplantation, T cells can recognize the antigenic peptides presented by the presenting cells of the recipient (indirect pathway) or of the donor (direct pathway). Since the direct presentation pathway is particularly involved during acute rejection, the inventors studied the impact of this type of recognition on the TCRβ repertoire. By virtue of the invention, which makes it possible to add a quantitative dimension to the qualitative alterations in the length distribution for the CDR3 region, it appeared that this distribution was not altered in the direct recognition, although the T cells are selected as a function of their Vβ segment. In addition, by following the regulation of expression of the TCR as a marker of activation, it is shown that a large proportion of naïve T cells can recognize “foreign” presenting cells, a phenomenon which makes it possible to understand more clearly the magnitude of the direct response and its involvement in acute rejection.

Analysis of the TCR repertoire makes it possible to evaluate the diversity of a T lymphocyte population and to monitor the evolution thereof under various experimental conditions or during the progression of a given pathological condition. It is thus possible to demonstrate restrictions (absence of certain Vβ families) or alterations in the repertoire (modification of the CDR3 length distribution in a Vβ family).

Analysis of the TCR receptor makes it possible to evaluate the diversity of a T lymphocyte population and to monitor the evolution thereof under various experimental conditions or during the progression of a given pathological condition. It is thus possible to demonstrate restrictions (absence of certain Vβ families) or alterations in the repertoire (modification of the CDR3 length distribution in a Vβ family).

Various techniques have been developed in order to analyze the CDR3 region and/or to quantify the variations in the repertoire at the level of the Vβ chain of the TCR. Currently, quantitative analysis of the TCR can be carried out by various methods: the RNAse Protection Assay, which has the disadvantage of being technically difficult, quantification from a competitive PCR or CD3, which does not distinguish between the various Vβ families, and anti-Vβ monoclonal antibodies. For quantitative analysis, many monoclonal antibodies directed against the various variable regions of the TCRαβ are available in humans and in mice and allow analysis of expression at the protein level. However, in rats, only three antibodies are available and effective, which limits the value of this method. In addition, analysis only at the protein level does not take into account the selection events occurring in the CDR3 region, which is the basis of the specificity of the antigen recognition. In addition, the percentage expression of a Vβ protein is not representative of the corresponding Vβ transcript percentage. In fact, such a quantification with antibodies runs the risk of underestimating the number of TCRs since they are internalized after activation. Other techniques, based on PCR amplification of the variable regions of the TCR using specific primers, make it possible to analyze the repertoire from genomic DNA. The amplification products thus obtained can be analyzed by autoradiography, cloned and sequenced.

The qualitative analysis of the TCR β-chain (second data) is based on studying the length distribution for the CDR3 hypervariable region of the TCR in each Vβ family of a given T lymphocyte population. A pool of resting T cells is characterized by a Gaussian distribution of the various lengths of the CDR3 in each Vβ family. The mobilization of specific T clones subsequent to recognition of an antigen is accompanied by the preferential use of certain TCRs composed of a Vβ family and having a particular CDR3 length.

The principle of the Immunoscope® technique consists of an amplification, by PCR, of the various CDR3 regions using a common 3′ primer placed in the Cβ segment and a 5′ primer specific for each Vβ family (see sequence listing). The products amplified are then subjected to an elongation step carried out with a second Cβ primer, labeled with a fluorophore. The fluorescent amplification products are separated as a function of their length by migration on a polyacrylamide gel. The migration profile is analyzed with a DNA sequencer coupled to the Immunoscope® software. An image of the CDR3 length distribution is thus obtained, with reference to FIG. 5, for each pair of primers. It is possible to observe between 1 and 11 amplification peaks, each corresponding to a particular size for the CDR3 region and each separated by three nucleotides. This analysis can be further refined if a primer specific for a Jβ segment is used instead of the common Cβ primer. As illustrated in FIG. 6, analysis of the distribution of the various CDR3 lengths makes it possible to identify possible oligoclonal expansions.

The Reperturb software makes it possible to compare the CDR3 length distribution in a given sample with that obtained in a reference individual (Gaussian profile, not altered). Each CDR3 length analyzed by the Immunoscope® software is translated into a probability of distribution, taking into account the area under the curve for each peak. For each one of them, the difference in absolute value between the sample (En) and the reference profile (Cn) makes it possible to obtain the percentage alteration of the sample relative to a Gaussian profile. This analysis therefore makes it possible to specify the level of alteration observed on an Immunoscope® profile. The variations obtained for each CDR3 expansion in a Vβ family will therefore be expressed as percentage alteration of a peak within this family. The sum of the alterations in all the Vβ families makes it possible to estimate the percentage of overall alteration of the TCR Vβ chain in a T cell population.

It is then necessary to perform the quantification of the Vβ transcripts, which can be approached using several techniques. In the case in point, the real-time quantitative PCR method can be used. This method, based on the use of the TaqMan® technology, can use the program known as “ABI Prism 7700 Sequence Detection System” which makes it possible, inter alia, to detect and measure the fluorescence emitted by the binding of a label (Sybr®Green) to double-stranded DNA molecules. The level of fluorescence, which is directly proportional to the amount of the product in a well, is collected in the course of each amplification cycle. In parallel with the samples, a standard, corresponding to a target sequence dilution range, can be used and will make it possible to establish a standard curve, which is used to deduce the amount of the target in each sample. In order to eliminate experimental variations between the various samples, these values are related to the measurement of the housekeeping gene HPRT.

In order to improve the specificity and the sensitivity of the PCR, the enzyme called AmpliTaq Gold® DNA polymerase is used. A modification in this enzyme makes it active only at high temperature, a temperature at which the DNA is completely denatured. In order to avoid a contamination with other PCR products, AmpErase® uracil-N-glycosylase (UNG) is added to the reaction mixture at the time of assaying. This enzyme, which is only active below 60° C., acts by hydrolyzing the uracil bridges in single- or double-stranded DNA containing uracil bases and has no activity on DNA containing thymidines. These uracil bridges are generated by the introduction of dUTP bases into the reaction mixture. During the amplification, an initial step of 2 minutes at 50° C. activates this amperase and makes it possible to eliminate possible contaminants derived from the preceding amplification.

The standards are prepared from reverse-transcribed RNA originating either from splenocytes from a naïve animal, or from T lymphocytes derived from the blood of healthy individuals, in humans. For the preparation of each Vβ standard, the amplification is carried out in a conventional thermocycler. The PCR products are then loaded onto an agarose gel and the band of interest is extracted. The concentration of the PCR product is estimated through the OD₂₆₀ value and then, as a function of the molecular mass, the number of copies per ml is calculated. Dilutions of each Vβ, Cβ or HPRT standard at 10⁷, 10⁶, 10⁵, 10⁴, 10³ and 10² copies per well are then prepared in order to obtain a concentration range which covers that of the samples.

In order to validate each standard, a second PCR is carried out, this time in the ABI PRISM 7700 Sequence Detector in the presence of the Sybr®Green fluorescent label. The PCR products are then loaded onto an agarose gel, which makes it possible to control the decreasing reduction in each band as a function of the dilution and also the size of the PCR product. In parallel, the increase in fluorescence intensity, which is followed as a function of time, is linked to the number of copies of DNA contained in each dilution. This makes it possible to verify the accuracy of the dilutions for the standard, the PCR yield and the absence of contamination.

Once validated, these standards make it possible to deduce the number of copies of the target in each sample, tested in duplicate, by relating the level of fluorescence to the standard straight line. This straight line is only taken into account if the efficiency of the PCR is close to 100% (slope=−3.3) and if the coefficient of correlation is greater than 0.95.

It is then a question of combining the qualitative and quantitative analyses so as to give a quantitative dimension to the alterations in the CDR3 length distribution. This information can make it possible to evaluate the representativeness of an oligoclonal expansion in a given population and can also make it possible to visualize modifications in the repertoire which are not associated with alterations in the CDR3 region.

The invention will preferably use a computer program developed in order to obtain an image containing all the information relating to the repertoire studied. The Matlab® software can be used for this purpose since it thus enables the flexible management of a very large volume of data and an imaging which is sufficiently clear and informative. For each Vβ family, the percentage alteration relative to the Gaussian profile is measured by the Gorochov method. By virtue of the Matlab® software, these values are combined with the first (quantitative) data so as to obtain a 3D representation illustrating both the qualitative and quantitative variations in the Vβ transcriptome, in which the height of the peaks illustrates the amount of corresponding Vβ transcripts, while the percentage alterations are represented by a color code.

On the graph in FIG. 9, the following appear:
along the x-axis, each one of the Vβ families;
along the y-axis, the quantitative data (i.e. the relative size of each Vβ family);
along the Z-axis, the various possible lengths of the CDR3; and
according to a color code, a visualization of the alterations in the repertoire relative to a Gaussian profile.

The color code can comprise a series of colors, for example from green to red in the direction of increasing alteration, i.e. with increasing distance from 0% alteration. In the case in point, it will involve various levels of gray, the gray becoming darker and darker as the alteration increases.

Thus, the Vβ transcripts, which may or may not be altered, are expressed according to a three-dimensional graph exhibiting, along the x-axis, each Vβ family and, along the y-axis, the quantitative data obtained by TaqMan (TaqMan PCR for Cβ and for each Vβ mRNA species). The percentage alteration for each Vβ relative to a Gaussian profile is visualized according to a color code (an alteration “range” corresponds to each color).

The method will be implemented by means of an installation which makes it possible to analyze the T lymphocyte receptors of an organism, illustrated diagrammatically in FIG. 10. This installation will comprise automated means for amplifying the CDR3 region of each of the Vβ genes of the T lymphocyte receptors associated with the organism (obtaining the second values), and for quantifying each Vβ gene (first data). It may, for example, be a machine (2) such as the TaqMan® device, which makes it possible, by virtue of following the amplifications in real time, to quantify the Vβ transcripts as they are amplified: the quantitative values thus obtained make it possible to compare amounts of transcripts of a certain Vβ family with amounts of transcripts of another Vβ family (example of device: ABI Prism 7900 from the company Applied Biosystems).

The installation will also comprise automated means for measuring the relative amounts of each length of the CDR3 region in each Vβ family and the ratio of these amounts to predetermined values, so as to obtain second data. It may be a sequencer (4) which makes it possible to determine the relative part of each transcript having a particular size for CDR3 in a given Vβ family, but which does not make it possible to compare the Vβ families with one another: (for example, the Magabace capillary sequencer from the company Amersham pharmacia biotech).

Finally, the installation will comprise automated means for representing, in a four-variable diagram, the first and second data as a function of the Vβ genes and of the length of the CDR3 regions. It may be a computer (6) controlled by a program developed, for example, using the abovementioned Matlab® software. The development of such a program in itself is within the scope of those skilled in the art and will not be detailed here. Optionally, this program will be able to control other steps in the progression of the method, or even the entire method. The diagram will be represented on an information medium, such as a computer screen or a paper medium (8).

This study can be carried out in the course of the kinetics of an immune response in various situations.

In this context of implementation, as in others, the invention makes it possible to carry out more thorough analyses than with the methods of the prior art. The invention makes it possible to obtain a general and a three-dimensional view of the size of the pool of T cells using a particular Vβ rearrangement and therefore makes it possible to determine the quantitative importance of the Vβ families selected during the immune response.

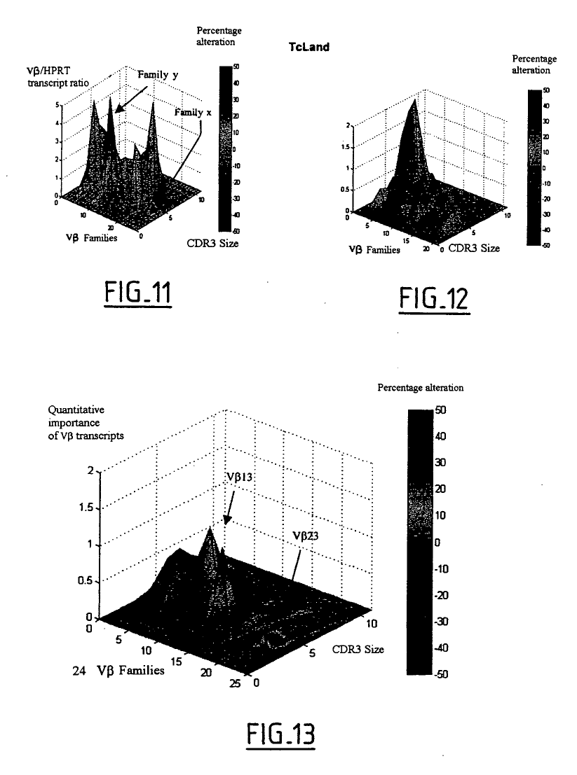

Thus, with reference to FIG. 11, which gives an example of the appearance of a diagram not associated with particular biological circumstances, it appears that some families, although highly altered relative to the Gaussian profile, are only weakly represented at the quantitative level and therefore do not appear to have a predominant role (example: family x). The invention also makes it possible to differentiate a resting situation from a situation in which the T cells are activated in a polyclonal manner. Specifically, the graph shown in FIG. 12 is derived from a model in which T cells are activated in a polyclonal manner, and a very strong mobilization of certain Vβ families which, nevertheless, exhibit a Gaussian distribution for the CDR3 lengths (which corresponds to 0% alteration) is then observed. Thus, this technique makes it possible, inter alia, to visualize polyclonal. activations (ConA, superantigen, direct presentation pathway) which would not have been detected by conventional tools. It is therefore a powerful means for monitoring the changes in the repertoire in the course of various immune responses.

In fact, the graph is derived from a model in which T cells are activated in vitro, and a very strong mobilization of certain Vβ families is observed. On this same scale, a control sample would give a green (light gray) profile since all the families exhibit a CDR3 length distribution which is Gaussian (which corresponds to 0% alteration) and which is flat since all the Vβ families are weakly represented. It is therefore a powerful means for monitoring the changes in the repertoire in the course of various immune responses.

FIG. 13 illustrates the diagram resulting from a second embodiment of the invention. The invention is here used in the context of a study carried out on patients who are suffering from melanoma and who have been vaccinated. The Vβ13 family appears to be particularly involved in the anti-melanoma response insofar as a peak appears which is red (dark gray), and therefore distant from the Gaussian profile characteristic of a normal response, and quantitatively important. Another family appears to have a less important role: the Vβ23 family, which is perturbed but relatively unimportant in quantitative terms. In addition, an unresolved peak appears in the region of the first Vβ families. This unresolved peak might correspond to an effect of the vaccination. Further studies will make it possible to determine this.

Described below is another embodiment in which the method of the invention is completed by a step consisting of isolating the clones exhibiting altered Vβ families.

The specific alterations in the repertoire which are detected using the invention are, most commonly, not detectable by conventional techniques of flow cytometry. In fact, while the invention makes it possible to detect an alteration and an accumulation of transcripts for a given Vβ family, flow cytometry only makes it possible to detect the membrane-bound expression of the Vβ protein. Now, surface protein expression is transient and does not make it possible to detect a modification in the transcriptional activity of a cell at a precise moment.

As has been seen, the invention thus makes it possible to detect a modification in transcriptional activity for a given family. Once the family has been detected, it is possible to extract it from the rest of the lymphocyte population in order to study the specific characteristics thereof (phenotype, activation, etc.). If these characteristics were studied in the total population, they would be masked by the sum of the characteristics of all the other T cell populations.

The present method of representation therefore makes it possible to identify particular populations involved in the immune response studied. These T cells, bearing a TCR composed of a particular Vβ family, can then be isolated by flow cytometry using anti-Vβ antibodies, for example, which makes it possible to characterize the clones bearing specific (qualitative and/or quantitative) alterations in the TCR β-chain repertoire.

After implementation of the invention as set out in the preceding embodiments, including the step for producing the diagram, the cells expressing the various Vβ families characterized by oligoclonal expansions and/or large amounts of messenger RNAs are extracted by flow cytometry using antibodies directed against the various Vβ families.

Thus, each Vβ family before sorting represents less than 5% of the Vβ repertoire. After sorting, it is possible to work on a given population which is very pure since the purity obtained is greater than 98%.

Several successive rounds of sorting are possible and therefore several T cell clones can be isolated from the same cell extract since, after selection of a particular T cell population, the remaining population comprises all the other T cell types. This same cell extract which has made it possible to isolate a first T cell population may therefore also serve as a basis for extracting another Vβ family. The possibilities and contributions of this technique are therefore very considerable and improve the basic technique of the invention. Once the number of cells extracted has been determined, the cells are used for phenotypic and transcriptional studies.

These studies are fundamental since they will make it possible to determine the role of a lymphocyte clone in a given situation. This determination of the role of T cell clones is very important insofar as, if the clone identified proves to have a cytotoxic role (viruses such as HIV, CMV or EBV; autoimmune diseases), it will be necessary to inhibit it in the context of immunotherapeutic strategies. Conversely, if this clone proves to have a protective role (transplantation, immunosuppressor treatments), it should be stimulated in order to obtain a beneficial effect.

To implement this technique, a flow cytometer may be used which makes it possible to isolate the T cells revealed by the technology according to the invention, after labeling of the T cell populations considered with an anti-Vβ antibody, such as that marketed under the name “Facscalibur” by the company Beckton Dickinson.

As illustrated by FIG. 14, the invention also relates to a method for identifying, in particular T lymphocyte populations, genes, molecules and mechanisms involved in physiopathological processes.

The method of analysis according to the invention is carried out and the T cell populations for which the TCR transcripts are strongly represented among all the transcripts of the total T cell population can be isolated, and the phenotype of these cells can be compared with respect to the phenotype of the cells of the general population, thus making it possible to demonstrate the pathways of response to stimuli.

Of course, many modifications may be introduced into the invention without departing from the context of the attached claims. Thus, on the diagram, the variable represented by differences in local appearance of the diagram may be one which is different from that corresponding to the second data.

The difference in local appearance may be illustrated other than by colors or shades of gray. It may, for example, be various types of hatchings or other types of signs.

The invention also relates to each primer for amplifying the CDR3 regions, as indicated in the sequence listing.

1. A method for analyzing the activation and mobilization of T cells through an analysis of the T lymphocyte receptors of an organism, characterized in that it comprises the steps consisting in:
determining the number of transcripts for each Vβ gene, so as to obtain first data;
amplifying the CDR3 region of the T lymphocyte receptors for each of the Vβ genes associated with the organism;
separating the various lengths of the CDR3 of the T lymphocyte receptors for each of the Vβ genes associated with the organism and estimating the proportion of transcripts for each length of the CDR3 in each Vβ family, so as to obtain second data;
representing, in a four-variable diagram, the first and second data as a function of the Vβ genes and of the length of the CDR3 regions.

2-11. (canceled)

12. The method as claimed in claim 1, characterized in that at least one of the variables is represented without being associated with a dimension in space.

13. The method as claimed in claim 12, characterized in that the variable which is not associated with a dimension in space corresponds to the second data.

14. The method as claimed claim 1, characterized in that at least one of the variables is represented by differences in local appearance of the diagram.

15. The method as claimed in claim 14, characterized in that the differences in local appearance are color differences.

16. The method as claimed claim 1, characterized in that the first data are represented in the vertical direction.

17. The method as claimed claim 1, characterized in that it comprises, after determination of the first and second data, the steps consisting in choosing a Vβ gene as a function of the results from the first two steps, and in extracting from the total T cell population the T cells bearing a TCR consisting of this preselected Vβ family.

18. The method as claimed in claim 1, characterized in that at least some of the steps are carried out in an automated fashion.

19. A computer program for analyzing the T lymphocyte receptors of an organism, characterized in that it is capable of controlling the implementation of at least the representation step of the method as claimed in claim 1.

20. An installation for analyzing the T lymphocyte receptors of an organism, characterized in that it comprises:
automated means for amplifying the CDR3 region of the T lymphocyte receptors for each of the Vβ genes associated with the organism;
automated means for measuring the amounts of each Vβ gene, so as to obtain first data;
automated means for measuring, for each Vβ gene, the relative amount of each CDR3 region with respect to all the CDR3 regions associated with a Vβ gene, so as to obtain second data; and
automated means for representing, in a four-variable diagram, the first and second data as a function of the Vβ genes and of the length of the CDR3 regions.

21. An information medium exhibiting a diagram analyzing the T lymphocyte receptors of an organism, characterized in that it comprises the following four variables:
the Vβ genes associated with the organism;
the length of the CDR3 regions of the T lymphocyte receptors;
first values representing the amount of transcripts of each Vβ gene in the form of relative or absolute amount;
second data representing, for each of the lengths of the CDR3 region in each Vβ gene, the difference between the proportion of transcripts corresponding to a CDR3 region of a Vβ gene in a given sample compared to the transcripts corresponding to the same CDR3 region of the same Vβ gene in a reference sample which is a sample exhibiting a Gaussian distribution for CDR3 lengths.
